(19) United States
(12) Patent Application Publication
(10) Pub. No.: US 2009/0081284 A1
(43) Pub. Date: Mar. 26, 2009

(54) THERAPEUTIC AGENT FOR ALLERGY CONTAINING LIPOSOME HAVING OLIGOSACCHARIDE ON ITS SURFACE

(75) Inventors: Naoya Kojima, Kanagawa (JP); Hideki Narumi, Kanagawa (JP); Hajime Masumoto, Kanagawa (JP); Tomokatsu Iwamura, Kanagawa (JP); Akihito Kaneda, Kanagawa (JP)

Correspondence Address: OBLON, SPIVAK, MCCLELLAND MAIER & NEUSTADT, P.C., 1940 DUKE STREET, ALEXANDRIA, VA, 22314, US

(73) Assignee: TOKAI UNIVERSITY EDUCATIONAL SYSTEM, SHIBUYA-KU (JP)

(21) Appl. No.: 12/297,714

(22) PCT Filed: Oct. 20, 2006
(86) PCT No.: PCT/JP2006/320937

§ 371 Date: Dec. 8, 2008

(30) Foreign Application Priority Data
Apr. 20, 2006 (JP) 2006-117122

(51) Int. Cl. A61K 39/36; A61K 9/127; A61P 37/08; A61K 39/35

(52) U.S. Cl. 424/450; 424/275.1

(57) ABSTRACT

An object of the present invention is to provide a therapeutic agent for allergy which realizes enhancement of therapeutic efficacy and improvement in side effects such as anaphylaxis in a method for treating allergy by allergen administration (hyposensitization therapy).

That is, the present invention provides the therapeutic agent for allergy comprising a liposome having on its surface an oligosaccharide capable of binding to a lectin derived from an antigen presenting cell and composed of 2 to 11 sugar residues, wherein an allergen is encapsulated in said liposome.

### TECHNICAL FIELD

The present invention relates to a therapeutic agent for allergy where an allergen is encapsulated in a liposome having oligosaccharides capable of binding to a lectin derived from an antigen presenting cell.

### BACKGROUND ART

Anti-histamine agents, chemical mediator releasing inhibitors, TXA2 receptor antagonists, LT antagonists and steroid agents have been mainly used for treating patients with allergy such as cedar pollen allergy, but all of the treatments are only symptomatic therapies not leading to a radical therapy.

Meanwhile, a radical therapy where an allergen (cedar pollen extract) in a trace amount as a therapeutic agent for allergy is subcutaneously injected once a week or two weeks and a loaded amount thereof is gradually increased by repeating the injection to finally suppress an occurrence of a symptom to the allergen (cedar pollens) is referred to as a hyposensitization therapy. However, the conventional therapeutic agent composed only of the allergen as an ingredient has only a weak action, its effective rate is low and it is necessary for obtaining its effect to be administered for a long time, e.g., several years. Thus, the hyposensitization therapy is not clinically employed frequently even though it is a radical treatment. In addition, there are problems of side effects such as increase of IgE production or occurrence of anaphylaxis in patients, since the allergen is administered to the patients with allergy (in the Example of the present invention, it has been confirmed that the administration of the allergen alone enhances an allergic response). A therapeutic agent for the allergy which overcomes such problems in these therapeutic agents for the allergy is highly useful.

A mechanism of allergy treatment in the hyposensitization therapy is unknown. However, it is believed that if the production of cytokines from Th2 cells in response to an antigenic stimulation can be specifically inhibited (improvement of a balance between a Th2 response and a Th1 response) and if the production of antigen-specific IgE which causes the allergic symptom can be inhibited (improvement of a balance of immunoglobulin class productions), then it can be expected to enhance the usefulness of the hyposensitization therapy. Therefore, the hyposensitization therapy is fundamentally different in idea from a vaccine therapy where the enhancement of usefulness can be expected by inducing humoral immunity and cellular immunity, although they share a common concept that the immunity is induced by an antigen.

Uchida et al. have found that the production of IgE is inhibited by administering a liposome binding an allergen on its surface (Nonpatent Literature 1). However, the side effect such as anaphylaxis is concerned because the allergen is exposed on the surface of the liposome. Gangal et al. have disclosed that by encapsulating the allergen in the liposome, the production of the allergen specific IgE is inhibited and the production of IgG is induced with inhibiting anaphylaxis (Nonpatent Literature 2). However, these do not analogize the usefulness of the liposome of the present invention which is incorporated in an antigen presenting cell via a mannose receptor. The effect of the general therapeutic agents for allergy where the allergen is encapsulated in the liposome (hyposensitization therapy) is still insufficient. This is as confirmed in animal experiments by the present inventors (see Examples 5 and 6 described later).

It has been reported that the liposome coated with macromolecular polysaccharides such as mannan developed as adjuvants for a vaccine and an immunotherapy has a potent ability to induce the cellular immunity (Patent Document 1, Nonpatent Literature 3). However, mannan is a mixture of polymannoses having different sizes, and is known to have a strong toxicity for living bodies (Nonpatent Literature 4). Thus, mannan is not suitable for pharmaceuticals. That is, mannan is a polysaccharide composed of 50 to 100 mannose residues and is heterogeneous in molecular weight, and the binding pattern of sugars is structurally unknown. It has been also known that when this polysaccharide is inoculated in animals an antibody is produced (having an antigenicity) and also it has the strong toxicity as described above.

Meanwhile, Mizuochi et al. have reported that the toxicity and the antigenicity of the sugar are removed and the effect as the vaccine is enhanced by encapsulating an antigen in the liposome having on its surface an oligosaccharide which is composed of 2 to 11 sugar residues and is capable of binding to a lectin derived from the antigen presenting cell (Patent Document 2). In this Patent Document 2, it has been also disclosed that a cellular immunity against the antigen encapsulated in the liposome having the oligosaccharide on its surface can be efficiently induced.

It is believed that the liposome having the oligosaccharide on its surface induces antigen specific T cells activation and cytokines derived from Th1 cells by being phagocytosed by the antigen presenting cell via the mannose receptor and presenting the antigen via an MHC class I or II molecule. However, the induction of an immune response via the mannose receptor and the MHC molecule does not necessarily induce only the immune response of Th1 type, and it is known that the cytokine derived from Th2 cells is induced in antigen presentation via the mannose receptor and the MHC class II molecule (Nonpatent Literatures 5 and 6). Th2 responses to an allergen are observed in the patients with allergy. Thus, whether it enhances the usefulness that the liposome having the oligosaccharide on its surface in Patent Document 2 is practically applied to the allergy therapy including the hyposensitization therapy has been thrown into doubt.

It has been also known that the inhibition of IgE production which is the most important in an allergy treatment is not directly associated with the induction of the Th1 type response responsible for a cellular immunity (Nonpatent Literature 7). Thus, it has been unknown whether the liposome having an oligosaccharide on its surface inhibits the IgE production or not.

Patent Document 1: International Publication WO92/04887 Pamphlet

Patent Document 2: Patent No. 2828391

Nonpatent Literature 1: Uchida et al., J. Immunol., 169:4246-4252, 2002

Nonpatent Literature 2: Gangal et al., Asian Pac. J. Allergy Immunol., 16:87-91, 1998

Nonpatent Literature 3: Noguchi et al., J. Immunol., 143:3737-3742, 1989

Nonpatent Literature 4: Mikami et al., The 15th Carbohydrate Symposium Proceedings, 43-44, 1993

Nonpatent Literature 5: Apostolopoulos et al., Proc. Natl. Acad. Sci. USA, 92(22):10128-10132, 1995

Nonpatent Literature 6: Apostolopoulos et al., Vaccine 14(9):930-938

Nonpatent Literature 7: Uchida et al., Curr. Drug Targets Immune Endocr. Methanol. Disord., 3:119-135, 2003

### DISCLOSURE OF INVENTION

### Problem to be Solved by the Invention

An object of the present invention is to provide a therapeutic agent for allergy which realizes enhancement of therapeutic efficacy and improves in side effects such as anaphylaxis in a method of treating the allergy by allergen administration (hyposensitization therapy).

### Means for Solving Problem

As a result of an extensive study to solve the above problems, the present inventors have found that a Th1 type response and a Th2 type response are highly balanced and IgE production is highly inhibited by encapsulating an allergen in a liposome having on its surface an oligosaccharide capable of binding to a lectin derived from an antigen presenting cell. Also it has been found that a response to immunoglobulin is inhibited by encapsulating the allergen in the same liposome. The present invention is based on such findings.

The present invention includes the following embodiments [1] to [10]:

[1] A therapeutic agent for allergy comprising a liposome having on its surface an oligosaccharide which is capable of binding to a lectin derived from an antigen presenting cell and is composed of 2 to 11 sugar residues, and the liposome encapsulating an allergen;
[2] The therapeutic agent according to [1], wherein the oligosaccharide is composed of 3 to 5 sugar residues;
[3] The therapeutic agent according to [1] or [2], wherein the oligosaccharide is composed of the sugar residues including mannose;
[4] The therapeutic agent according to any one of [1] to [3], wherein the allergen is a pollen antigen;
[5] The therapeutic agent according to [4], wherein the pollen antigen is a cedar pollen antigen;
[6] The therapeutic agent according to any one of [1] to [5], applying by subcutaneous, intradermal or nasal administration, intradermal or nasal administration;
[7] A liposome having on its surface an oligosaccharide which is capable of binding to a lectin derived from an antigen presenting cell and is composed of 2 to 11 sugar residues, and the liposome encapsulating a pollen antigen;
[8] The liposome according to [7], wherein the oligosaccharide is composed of 3 to 5 sugar residues;
[9] The liposome according to [7] or [8], wherein the oligosaccharide is composed of the sugar residues comprising mannose; and
[10] The liposome according to any one of [7] to [9], wherein the pollen antigen is a cedar pollen antigen.

### EFFECT OF THE INVENTION

The therapeutic agent for allergy provided in the present invention has a high therapeutic efficacy as well as reduces a risk for side effects. Thus, by using it for the hyposensitization therapy, it becomes possible to achieve a radical treatment for an allergy which is safe and highly effective in a short time as compared with the conventional hyposensitization therapy.

### BRIEF DESCRIPTION OF DRAWINGS

FIG. 1 shows methods for tests of evaluating effects of inducing Th1 responses (Example 5: A) and inhibiting IgE production (B in Examples 6 and 8) in mice;

FIG. 2 shows a method for a test of evaluating therapeutic efficacy in mice (Example 7);

FIG. 3 shows a graph indicating results of measuring amounts of IFN-γ produced in culture supernatants after culturing in the presence of Cryj 1 spleen cells from mice treated with a test substance in Example 5;

FIG. 4 shows a graph indicating a ratio of measured antigen-specific IgG2a to IgG1 (IgG2a/IgG1) in serum from mice treated with the test substance in Example 5;

FIG. 5 shows a graph indicating results of measuring total IgE concentrations in serum obtained by collecting blood with time from mice treated with the test substance and administered with a mixture of Cryj 1 and alum in Example 6;

FIG. 6 shows a graph indicating results of measuring amounts of IL-5 produced in culture supernatant when spleen cells from mice treated with the test substance and administered with the mixture of Cryj 1 and alum were cultured in the presence of Cryj 1 in Example 6;

FIG. 7 shows a graph indicating results of measuring total IgE concentrations in serum from mice sensitized with Cryj 1 and alum and treated with the test substance in Example 7;

FIG. 8 shows a graph indicating results of measuring antigen-specific IgE in serum collected from mice sensitized with a cedar pollen extract and alum and treated with the test substance in Example 8;

FIG. 9 shows a graph indicating results of measuring antigen-specific IgG2a in serum collected from mice sensitized with the cedar pollen extract and alum and treated with the test substance in Example 8; and

FIG. 10 shows a graph indicating results of measuring total IgE concentrations in serum collected from mice sensitized with Cryj 1 and alum and treated with the test substance in Example 9.

### BEST MODES FOR CARRYING OUT THE INVENTION

A therapeutic agent for allergy of the present invention is characterized by containing a liposome having an oligonucleotide which is capable of binding to a lectin derived from an antigen presenting cell and is composed of 2 to 11 sugar residues on its surface, and an allergen encapsulated in the liposome.

In the present invention, the liposome means a molecule formed of lipid(s) and having a void inside it. In the present invention, the liposome may be multilamellar vesicles or unilamellar vesicle. These can be produced according to known standard methods. According to the standard methods, one type can be converted to the other type. For example, a multilamellar type liposome can also be converted to a unilamellar type liposome. A particle diameter of the liposome used in the present invention is not particularly limited, and the particle diameter may also be sorted by passing through a filter having a desired pore size. The preferable particle diameter is 50 nm to 3 μm.

The liposome used in the present invention, which has the oligosaccharide capable of binding to a lectin derived from an antigen presenting cell and composed of 2 to 11 sugar residues on its surface, has the oligosaccharide capable of binding to the lectin derived from the antigen presenting cell and composed of 2 to 11 sugar residues on its surface. This liposome is sometimes referred to as an oligosaccharide liposome in the present invention. Here, the antigen presenting cell means a macrophage, a dendritic cell and the like. The lectin derived from antigen presenting cells means a lectin present on a surface of the antigen presenting cell, such as the mannose receptor. In the present invention, the oligosaccharide composed of 2 to 11 sugar residues can be appropriately selected from those having a nature of binding to the aforementioned lectin. The sugar residues which compose the oligosaccharide may include monosaccharides such as D-mannose (D-Man), L-fucose (L-Fuc), D-acetylglucosamine (D-GlcNAc), D-glucose (D-Glc), D-galactose (D-Gal), D-acetylgalactosamine (D-GalNAc) and D-rhamnose (D-Rha), and mixed oligosaccharides of them may also be used. Among them, those composed of the sugar residues containing D-mannose are preferable, and among others, those composed of D-mannose and those composed of D-mannose and D-acetylglucosamine are preferable, and in particular, those composed of D-mannose are preferable. The oligosaccharides composed of D-mannose may include mannobiose (Man2), mannotriose (man3), mannotetraose (Man4), mannopentaose (Man5), mannohexaose (Man6) and mannoheptaose (man7). The binging mode of the sugar residues which constitute the oligosaccharide may include α1→2 bond, α1→3 bond, α1→4 bond, α1→6 bond and α1→4 bond, and they may be included alone or in combination of two or more. Each sugar chain may be bound linearly or two or more sugar chains may be bound to one sugar residue to form a so-called branched bond. A number of the sugar residues is 2 to 11, preferably 3 to 11 and among others 3 to 5. The oligosaccharide may include more specifically M3 (formula 1), M5 (formula 2) and RN (formula 3) composed of the structure shown by the following formulae. Among them, preferable are M3 (formula 1) and MS (formula 2), and more preferable is M3 (formula 1). In the following formula (3), mannose (Man) with α1→2 bond each independently may be present or may not be present.

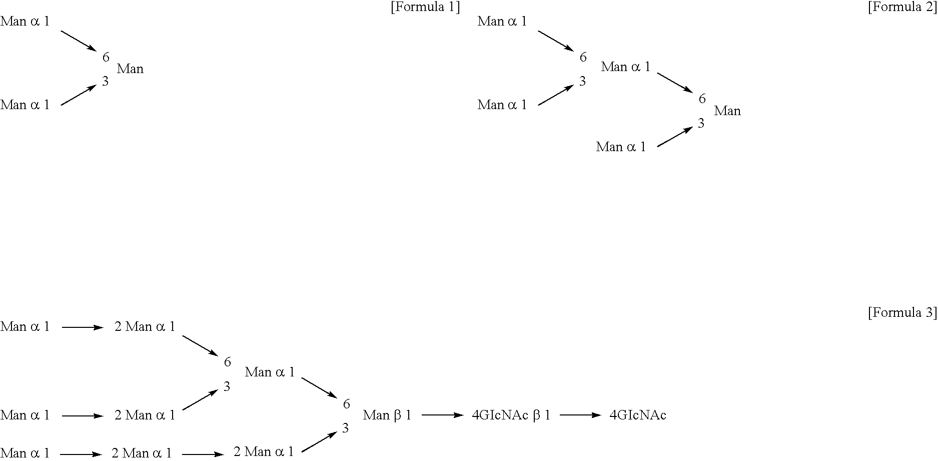

The amount of the oligosaccharide based on the amount of the liposome varies depending on the types of the oligosaccharide, the type of the allergen to be encapsulated and the combination structure of the liposomes, and is generally 0.5 to 500 μg based on 1 mg of the lipid which composes a liposome.

The lipid which composes a liposome may be the ordinary lipid known to compose a liposome, and can be used alone or combination of two or more. Examples of such a lipid may include lipids derived from natural products such as egg yolk, soybean or other animals and plants, those whose unsaturation degree is reduced by hydrogenation thereof or chemically synthesized lipids. More specifically, sterols such as cholesterol (Chol), 3β-[N-(dimethylaminoethane)carbamoyl]cholesterol (DC-Chol) and N-(trimethylammonioethyl)carbamoyl cholesterol (TC-Chol); phosphatidyl ethanolamines such as dipalmitoyl phosphatidyl ethanolamine (DPPE) and distearoyl phosphatidyl ethanolamine (DSPE); phosphatidyl cholines such as dipalmitoyl phosphatidyl choline (DPPC) and distearoyl phosphatidyl choline (DSPC); phosphatidyl serines such as dipalmitoyl phosphatidyl serine (DPPS) and distearoyl phosphatidyl serine (DSPS); and phosphatidic acids such as dipalmitoyl phosphatidic acid (DPPA) and distearoyl phosphatidic acid (DSPA) may be included. Alphabets in parentheses after compound names exemplified here represent abbreviations of respective compounds, and these abbreviations are used below.

When the oligosaccharide is introduced into a liposome, an artificial glycolipid prepared by binding the oligosaccharide and the lipid can be used as described later. As a method for preparing the artificial glycolipid, the following method may be illustrated by an example using the above oligosaccharide. Any of the above oligosaccharides has one reduced terminal aldehyde group. Thus, to introduce the oligosaccharide into a liposome surface, this aldehyde group is reacted with the phospholipid having an amino group to form a Schiff base. Subsequently, the oligosaccharide can be bound to the lipid by reducing, preferably chemically reducing this Schiff base such as NaBH₃CN according to the standard method (Tsugio Mizuochi, Shishitsu Kogaku, pages 224-232, Industrial Research Center of Japan, Biotechnology Information Center). Here, the aforementioned lipid which composes a liposome can be used as the lipid. In particular, those containing phosphate ester or a C—P bond can be preferably used. The lipid to be bound does not necessarily contain phosphate, and the lipid such as sterol can be used. A bound product of the oligosaccharide and the lipid is referred to as the artificial glycolipid in the present invention.

In order to introduce the oligosaccharide into a liposome surface, either the following two methods can be employed when the aforementioned artificial glycolipid is utilized. When the artificial glycolipid is water-soluble and is not dissolved sufficiently in an organic solvent, for example when a bound product of RN and DPPE (RN-DPPE) is used as the artificial glycolipid, the following may be employed: an aqueous solution of these RN-DPPE is prepared, and mixed with the formed liposome, and then the resultant is incubated under a temperature, for example, at 4° C. to 80° C. (preferably the substance to be encapsulated is not degraded), room temperature or a phase transition temperature, for 0.5 to 120 hours, for example about 24 hours. On the other hand, when the artificial glycolipid is soluble in an organic solvent, the artificial glycolipid together with the lipid for constituting a liposome may be dissolved in an organic solvent in the process for producing a liposome followed by the forming process of a liposome according to the standard method. The oligosaccharide bound to the liposome surface can be examined by adding the lectins corresponding to the sugar to induce an aggregation reaction of the liposomes.

The therapeutic agent for allergy of the present invention is characterized in that the allergen is encapsulated in a liposome. The amount of the allergen to be encapsulated is desirably 0.1 to 500 μg against 1 mg of the lipid used for the liposome, but is not particularly limited, and can be appropriately regulated depending on an administration route. A form of the allergen to be encapsulated is not particularly limited, and can be purified natural allergens, synthetic peptides, recombinant proteins, crude extracts, polysaccharides and sugars, and mixtures, degraded products and modified products thereof. The allergen is not limited to natural products and synthesized products, and includes degraded fragments, recombinant proteins, peptides including T cell epitopes and synthesized peptides.

Types of allergens are not particularly limited as long as they are the substances which cause an allergy, and specifically include tree pollen allergens, grass pollen allergens, mite allergens, house dusts, animal allergens and food allergens, are not limited thereto and include the allergens newly identified. Among them, allergic antigens such as cedar pollens, pollens of ragweed, cocksfoot and tansy, foods such as rice, wheat, buckwheat, cow milk, egg yolk and egg white, animal skins such as dog hairs, cat hairs and feathers, and fungi such as Candida and Aspergillus can be preferably used. Representatives among them may include the allergic antigen of pollens (pollen antigens), particularly the allergic antigen of the cedar pollen (cedar pollen antigen).

The allergen can be prepared from natural products containing the allergen, for example, the pollen by purifying with an ordinary column work. Furthermore, in the preparation of the allergen, processes such as a process for removing, partially degrading, or modifying the sugar chain of the obtained allergen may be appropriately added. The allergen may be made by a method for preparing the recombinant protein by using a microorganism such as Escherichia coli, an animal cell or a plant cell, introducing an entire allergen gene and expressing it, or alternatively a method for synthesizing a peptide fragment containing a partial sequence or a T cell epitope by protein engineering or peptide syntheses.

When the therapeutic agent for allergy of the present invention is used as a therapeutic agent for the patients with cedar pollen allergy, the allergen suitable for being encapsulated in the oligosaccharide liposome can be a cedar pollen extract, or a Cryj 1 antigen, a Cryj2 antigen or a cedar pollen antigen newly purified or mixtures thereof. Among them, the cedar pollen extract and the Cryj 1 antigen are particularly preferable.

In the present invention, publicly known methods, for example, the methods (Vortex method and ultrasonic method) described in D. W. Deemer, P. S. Uster, “Liposome” ed. by M. J. Ostero, Marcel Dekker Inc., N.Y. Basel, pages 27-, 1983, an ethanol injection method, an ether method and a reverse phase evaporation method can be applied to the production of the liposome in which the allergen has been encapsulated, and these can be applied in combination.

The therapeutic agent for allergy of the present invention can treat or prevent the allergic symptom against the allergen encapsulated in the liposome by administering the therapeutic agent to a patient with allergy because it contains the liposome encapsulating the allergen as described above. Usefulness of the present invention can be confirmed with the methods described in Examples, the methods in Non-patent Literature 1 or the methods of Taniguchi et al. (Int. Arch. Allergy Appl. Immunol., 89: 136-142, 1989), but methods to confirm the usefulness of the present invention are not necessarily limited thereto. The allergic diseases to which the present invention is applied are the diseases in which the symptoms such as rhinitis, dermatitis, conjunctivitis, bronchitis, cough and sneeze are induced by the allergen such as tree pollen allergens, grass pollen allergens, mite allergens, house dusts, animal allergens and food allergens. Specifically, the allergic diseases are the diseases in which the symptoms such as rhinitis are induced by pollens of Japanese cedar, ragweed, cocksfoot and tansy, foods and beverages such as rice, wheat, buckwheat noodles, cow milk, egg yolk and egg white, epidermis such as dog hairs, cat hairs and feathers, and fungi such as Candida and Aspergillus, but the diseases are not particularly limited, and the allergic diseases induced by a newly identified allergen are included. Among them, the particularly preferable allergic disease is the pollen allergic disease, and the pollen allergic disease by Japanese cedar pollen may be included as the representative.

The therapeutic agent for allergy of the present invention can be orally or parenterally administered to a patient as a suspension in buffer such as saline or as a pharmaceutical composition mixed with a carrier or an excipient which is known publicly and pharmacologically acceptable. As an administration method when administered parenterally, a subcutaneous injection, an intradermal injection and an intramuscular injection are preferably used. A dosage form for the parenteral administration may include eye drops, ointments, injectable agents, plasters, suppositories, nasal absorbents, lung absorbents, percutaneous absorbents and topical releasing agents. In the formulation, human serum albumin, human immunoglobulin, α2-macroglobulin, amino acids and sugars can be added as stabilizers, and also alcohol, sugar alcohol, ionic surfactants and nonionic surfactants can be added as dispersants and absorption accelerators in the range in which a physiological activity is not impaired. Trace metals and organic acid salts can be added as needed. The present invention can be combined with a drug for the symptomatic therapy of allergy. A dosage per once of the therapeutic agent for allergy of the present invention can be appropriately determined in the range of 1 pg to 200 μg as the general amount of the allergen, but is not necessarily limited, and is appropriately selected depending on the condition of a patient, the administration route and the administration interval.

The present invention will be specifically described below based on Examples, but these are only for the exemplifications, and do not limit the present invention in any meanings.

### EXAMPLES

### Example 1

### Preparation of Artificial Glycolipid

600 μL Of distilled water was added to 2.5 to 5 mg of mannotriose (Man3) having a structure of Manα1→6 (Manα1→3) Man, which was stirred and dissolved to prepare an oligosaccharide solution. On the other hand, DPPE at 5 mg/mL was dissolved in a mixed solution of chloroform/methanol (volume ratio of 1:1) to prepare a DPPE solution. NaBH₃CN at 10 mg/mL was dissolved in methanol to prepare an NaBH₃CN solution. Subsequently, 9.4 mL of the DPPE solution and 1 mL of the NaBH₃CN solution were added in 600 μL of the oligosaccharide solution, and the mixture was stirred and mixed. This reaction mixture was incubated at 60° C. for 16 hours to generate an artificial glycolipid. This reaction mixture was purified by a silica gel column and a C18 reverse phase column to yield the artificial glycolipid M3-DPPE.

### Example 2

### Preparation of Extract and Purification of Allergen

An extract extracted from cedar pollens was prepared and Cryj 1 which was a major allergen in cedar pollen allergy was purified in accordance with the publicly known method (H. Yasuda et al., J. Allergy Clin. Immunol., 71:77-86, 1983; M. Sakaguchi et al., 45:309-312, 1990). The cedar pollens (150 g) were extracted with 0.125 M sodium hydrogen carbonate, and ammonium sulfate was added to concentrate the extract. The precipitate with ammonium sulfate was dialyzed against PBS (−). The dialyzed product was used as a cedar pollen extract. For the purification of Cryj 1, the precipitate with ammonium sulfate was dialyzed against 0.05 M Tris, pH 7.8. The dialyzed solution was applied onto a DEAE-Sephadex column and a fraction which passed straight through the column was collected. The collected solution was dialyzed against 10 mM acetate buffer pH 5.0 followed by being absorbed to CM-Sephadex. Elution was performed with 0.1 M phosphate buffer, pH 7.2, 0.3 M NaCl and 1 mM EDTA to yield a cedar pollen major antigen (SBP: sugi basic protein). SBP was dialyzed against 0.1 M acetate buffer, pH 5.0, and Cryj 1 and Cryj 2 were separated from SBP using a Mono S column, and finally 12 mg of Cryj 1 was yielded.

### Example 3

### Preparation of Liposome in which Cryj 1 or Cedar Pollen Extract has Been Encapsulated

Cholesterol, dipalmitoyl phosphatidyl chorine (DPPC), mannotriose dipalmitoyl phosphatidyl ethanolamine (M3-DPPE) produced in Example 1 were mixed at a molar ratio of 10:10:1, or cholesterol and dipalmitoyl phosphatidyl choline (DPPC) were mixed at a molar ratio of 1:1, the mixture was dissolved in 2 mL of chloroform, and a lipid film was made in a pear-shaped flask. Subsequently, 3.75 mg/mL of Cryj 1 or the extract (0.375 mg/mL of Cryj 1 contained-extract) obtained in Example 2 was added to the lipid film to make liposomes by vortex in a water bath at 40° C. Then, particle sizes of the liposomes were selected 5 times by using a particle size selector of an extruder with a filter of 1 μm applying the pressure in the range of 0.2 to 1 MPa. Subsequently, the liposome solution was collected by centrifugation, and then the antigen not being encapsulated in the liposome was removed by repeating suspension, centrifugation and removal of the supernatant three times. For the analysis of the obtained liposomes, the amounts of cholesterol and Cryj 1 were measured using Cholesterol E Test Wako (Wako Pure Chemical Industries Ltd., 439-17501) and Modified Lowry Protein Assay Reagent Kit (Pierce, 23240), respectively. A recovery rate of the lipid was nearly 100%, and the rate of Cryj 1 was 2 to 5%. The Cryj 1 concentration of the encapsulated mixture was correlated with the value of the analysis.

### Example 4

### Reactivity of Cryj 1-Encapsulated Liposome with Anti-Cryj 1 Immunoglobulin

In order to identify the reactivity of an immunoglobulin having a binding activity to Cryj 1 present in an outer aqueous phase (outer layer) of the liposome with Cryj 1 encapsulated in the liposome, sandwich ELISA was performed using the suspension of the liposomes encapsulating Cryj 1 produced in Example 3. First, an anti-Cryj 1 rabbit antibody (Hayashibara Biochemical Laboratories Inc., HBL-Ab-1-000) was immobilized on a bottom of a plate, and the liposome suspension or a free Cryj 1 solution not encapsulated in the liposome as a standard substance was reacted. The detection was performed using a peroxidase-labeled anti-Cryj 1 monoclonal antibody 053 (Hayashibara Biochemical Laboratories Inc., 0HBL-Ab-1-053P). As a result, the amount of Cryj 1 detected outside the liposome was 0.1% or less relative to the amount of Cryj 1 detected inside the liposome. Cryj 1 in the outer layer of the liposome was removed by washing three times upon preparing the liposome, and it can also be determined that Cryj 1 was not leaked from the liposome during the storage and the measurement. Thus, it can be expected that the risk of causing the side effect such as anaphylaxis by reacting immunoglobulin against Cryj 1 present in a patient with Cryj 1 in an administered drug is largely reduced by using the liposome encapsulating Cryj 1 compared with the case of using the solution of Cryj 1 which is not encapsulated in the oligosaccharide liposome.

### Example 5

### Identification of Th1 Type Immune Induction by Treatment with Oligosaccharide Liposome

Concerning abbreviations for the liposome inclusion bodies used in the following Examples, “Cryj 1/M3-L” indicates the liposome having the oligosaccharide (Man3) prepared using 3.75 mg/mL of Cryj 1 on the surface, and “Cryj 1/L” indicates the liposome having no oligosaccharide (Man3) and encapsulating 3.75 mg/mL of Cryj 1. “Cryj 1” indicates that Cryj 1 was directly administered without being encapsulated in the liposome.

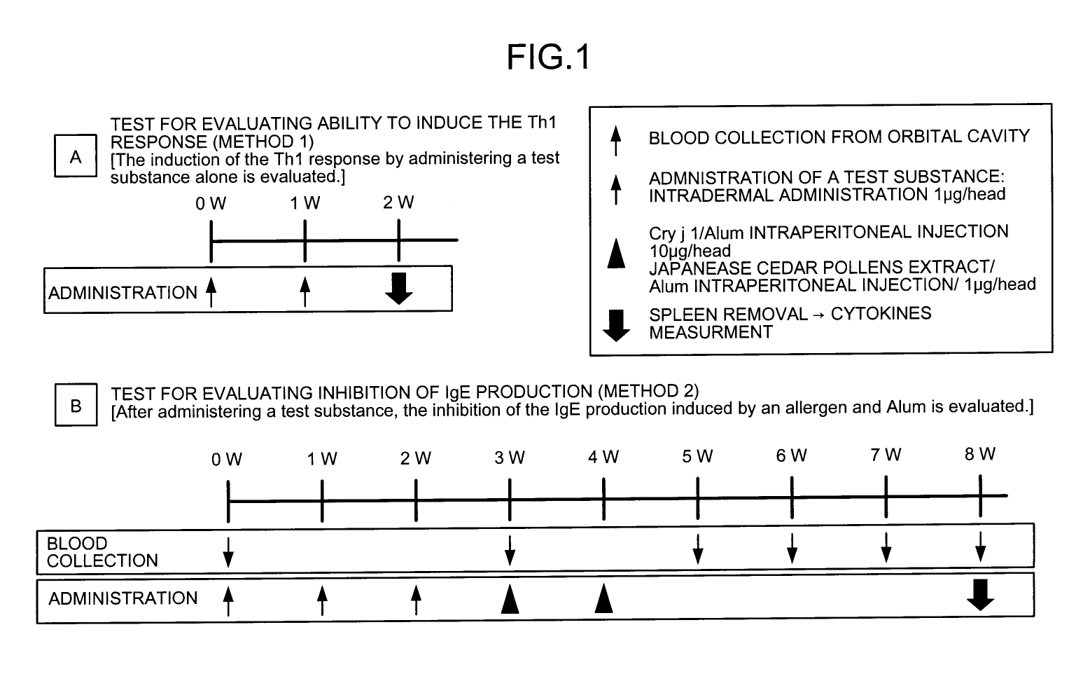

Cryj 1, Cryj 1/L or Cryj 1/M3-L (administered Cryj 1 as a protein amount: 2.5 g/head, intraperitoneal) was administered twice with one week interval to BALB/c mice aged 6 weeks. One week after the final treatment of the test substances, spleen was removed from each mouse, and a cell suspension (5×10⁶ cells/mL, RPMI 1640 medium) was prepared. The cell suspensions were cultured in the presence of Cryj 1 (final concentration: 50 μg/mL) in a CO₂ incubator for 72 hours, and a culture supernatant was collected from each cell suspension. IFN-γ (indicator for Th1 response) in the collected culture supernatant was measured by EIA. Blood was collected after the treatment with the test substance, and antigen-specific IgG2a (indicator for Th1 response) and IgG1 (indicator for Th2 response) in the blood were measured. These measured values were calculated to determine a Th1/Th2 balance, which was then used to evaluate a Th1 response induction effect of the test substance (FIG. 1A). As a control, PBS alone was administered in place of the above inclusion body and the evaluation was performed similarly.

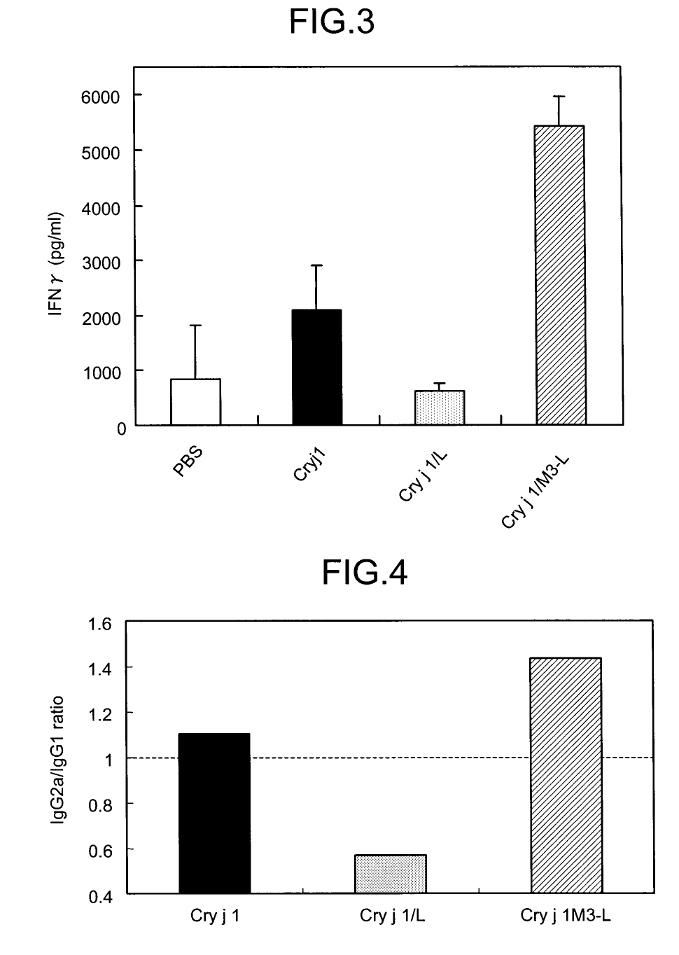

As a result, it has been demonstrated that the level of IFN-γ 1 in Cryj 1/M3-L treating group was largely increased compared with those in Cryj 1 treating group and Cryj 1/L treating group (FIG. 3). In addition, in the measurement ratio of antigen-specific IgG2a to IgG1 (IgG2a/IgG1) in each treating group, the Cryj 1/M3-L showed the high value compared with the value of the other treating groups (FIG. 4).

From these, it has been found that the allergen-encapsulated oligosaccharide liposome exhibits the high Th1 response induction effect in vivo compared with the treatment with the allergen alone and the allergen-encapsulated liposome, and is not accompanied with the induction of the Th2 response.

### Example 6

### Inhibitory Effect by Treatment with Oligosaccharide Liposome on IgE Production in Mice

Cryj 1, Cryj 1/L or Cryj 1/M3-L (administered antigen as the protein amount: 1 μg/head, intradermal administration) was administered three times with one week interval to BALB/c mice aged 6 weeks. One week after the final treatment of the test substances, a mixture of Cryj 1 and alum (administered antigen as the protein amount: 10 μg/head) was intraperitoneally administered twice with one week interval to boost the Th2 response. The blood was collected from orbital cavity before the treatment with the test substance, and before and after the administration of the mixture of Cryj 1 and alum to obtain serum. The blood was also collected with time after the treatment with Cryj 1 and alum. And then, the spleen was removed from each mouse, and homogenated to prepare a spleen cell suspension (5×10⁶ cells/mL, RPMI 1640 medium). The spleen cell suspension from each mouse was cultured in the presence of Cryj 1 (final concentration: 50 μg/mL) in the CO₂ incubator for 72 hours, and a culture supernatant was collected. Interleukin (IL)-5 (indicator for Th2 response) in the collected culture supernatant and the amount of total IgE in the serum were measured by EIA (FIG. 1B).

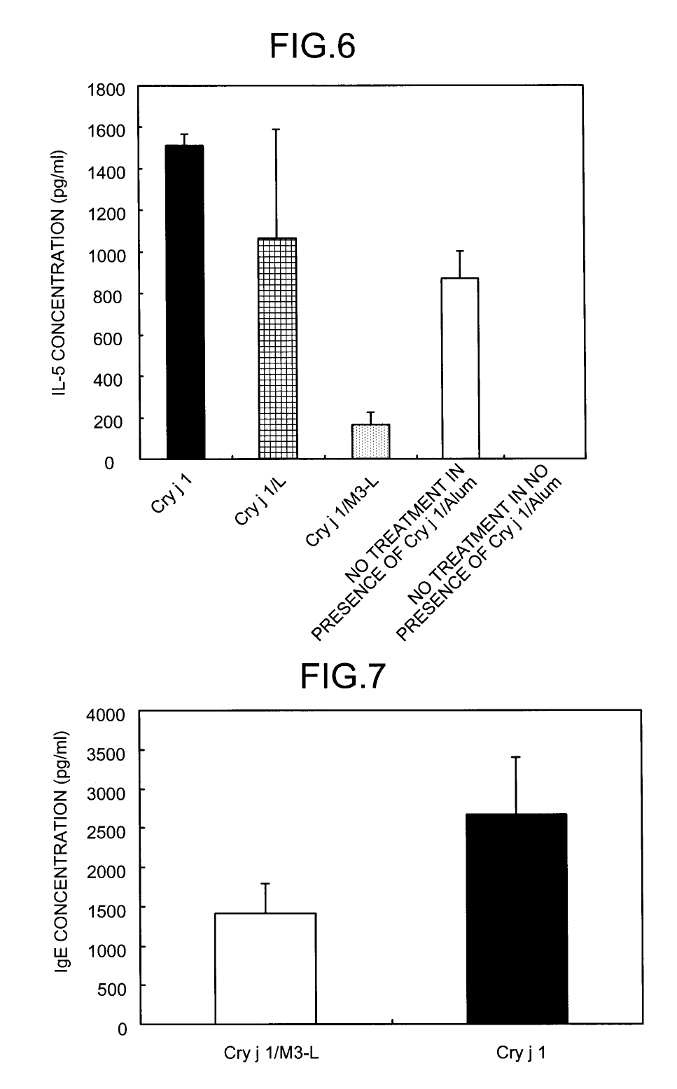

As a result of measuring the amount of produced IL-5 in the spleen cells, it was demonstrated that the amount of IL-5 production was inhibited in the Cryj 1/M3-L treating group even though the same amount of IL-5 production as those in the Cryj 1 alone treating group and a non-treating group was observed when Cryj 1/L was administered (FIG. 6).

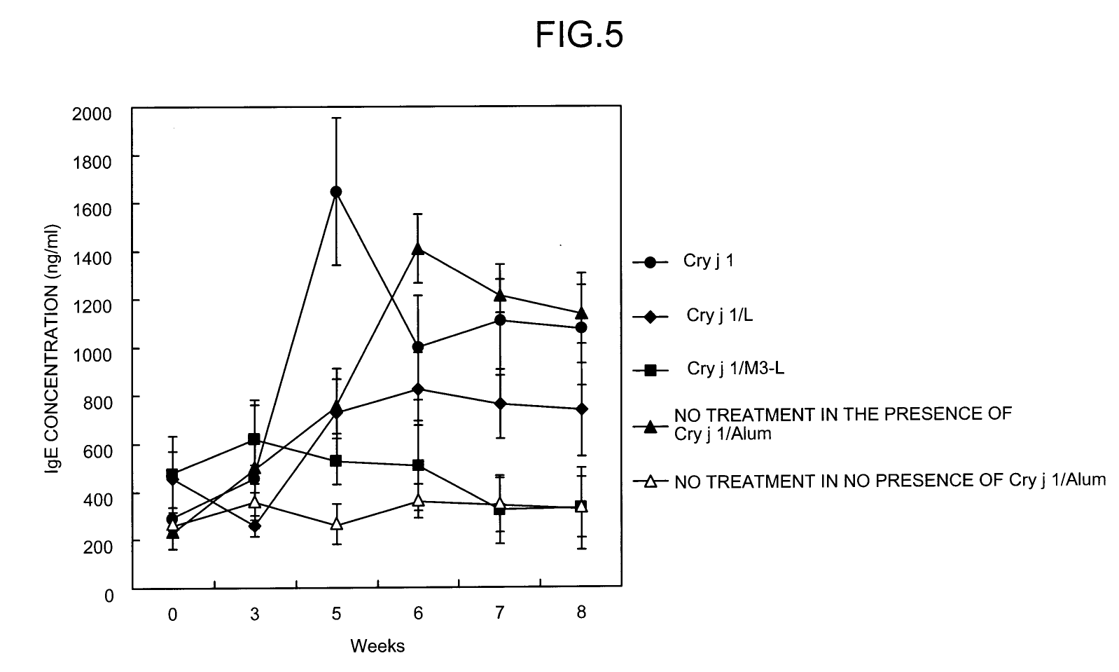

Furthermore, the amount of IgE production which increased in the non-treating group after administering the mixture of allergen and alum was inhibited in the Cryj 1/M3-L finally to the same level as in the group not administered with the mixture of allergen and alum, whereas it was not inhibited in the Cryj 1 treating group and the Cryj 1/L treating group (FIG. 5).

From these, it has been found that the oligosaccharide liposome encapsulating the allergen has the effects to more potently inhibit the Th2 response and the IgE production when exposed to the allergen, compared with the publicly known administration of the allergen alone and the administration of the conventional liposome encapsulating the allergen. The oligosaccharide liposome encapsulating the allergen of the present invention can be expected to inhibit the occurrence of the allergic symptoms of patients with allergy such as pollen disease.

### Example 7

### Therapeutic Effect on Allergy in Mice

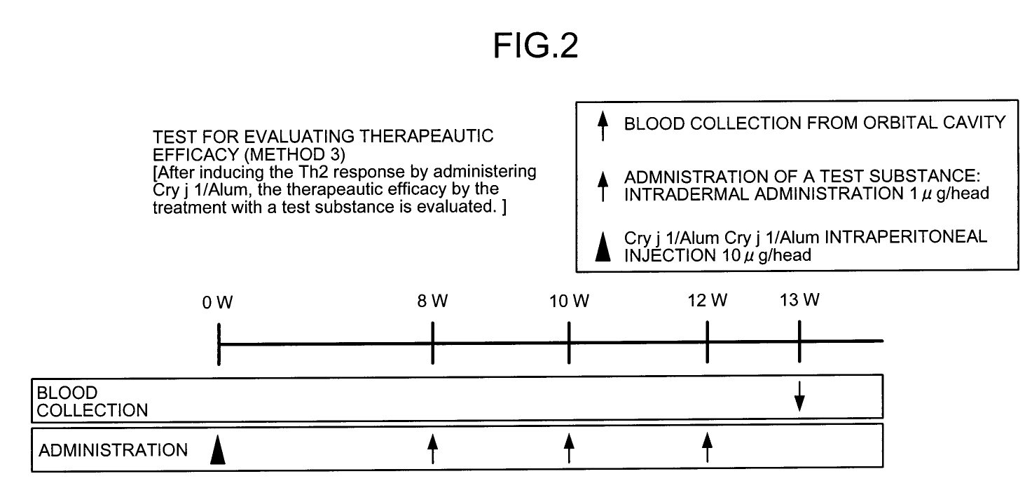

The Th2 response was induced by intraperitoneally administering the mixture of Cryj 1 and alum once to BALB/c mice aged 6 weeks. Eight weeks after the induction, Cryj 1 or Cryj 1/M3-L (administered Cryj 1 as a protein amount: 1 μg/head, intradermal administration) was administered three times with one week interval. One week after the final treatment with the test substance, the blood was collected, and the amount of total IgE in the serum from each mouse was measured by EIA (FIG. 2). Cryj 1/M3-L inhibited the IgE production compared with the Cryj 1 alone treating group (FIG. 7). It was found that, the oligosaccharide liposome encapsulating the allergen improved the condition where an allergic state such as a shift to the Th2 response had been formed in the mouse and, had the inhibitory effect on the IgE production.

### Example 8

### Inhibitory Effect of Treatment with Oligosaccharide Liposome Encapsulating Cedar Pollen Extract on IgE Production in Mice

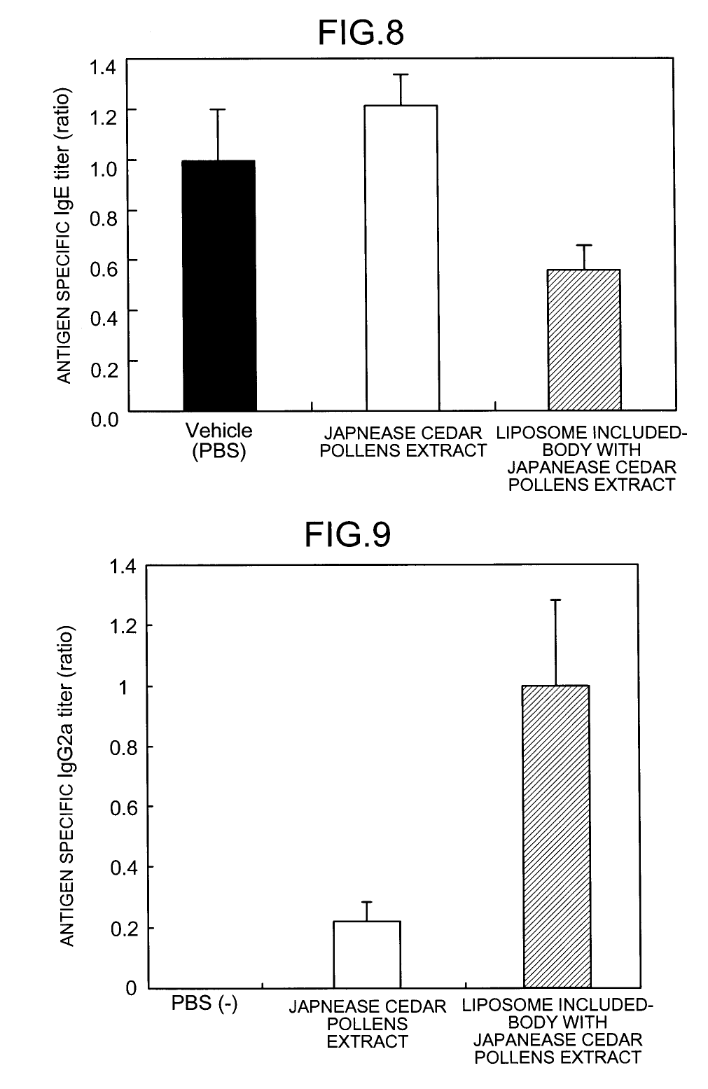

PBS (−), the cedar pollen extract or the oligosaccharide liposome encapsulating the cedar pollen extract was administered three times with one week interval to BALB/c mice aged 6 weeks (the amount of the administered antigen as the protein [total protein amount]: 0.3 μg/head, intradermal administration). One week after the final treatment with the test substance, a mixture of the cedar pollen extract and alum was intraperitoneally administered twice with one week interval to all of the mice (the amount of the administered antigen as the protein: 1 μg/head) to induce the Th2 response. The blood was collected from the orbital cavity before the treatment with the test substance, and before and after the administration with the mixture of the cedar pollen extract and alum to obtain the serum from each mouse (FIG. 1b). The amounts of antigen-specific IgE and IgG2a in the obtained serum were measured by EIA (FIGS. 8 and 9).

The IgE production which increased after the administration with the mixture of the cedar pollen extract and alum in the PBS (−) treating group was inhibited in the group treated with the oligosaccharide liposome encapsulating the cedar pollen extract, whereas it was not inhibited in the group treated with the cedar pollen extract (FIG. 8). It was identified that the more amount of antigen-specific IgG2a in the serum was produced in the group treated with the oligosaccharide liposome encapsulating the cedar pollen extract than in the group treated with PBS (−) or the cedar pollen extract (FIG. 9).

In view of the foregoing, it was found that the oligosaccharide liposome encapsulating the cedar pollen extract had the effect of inhibiting the IgE production similarly to Example 9. Therefore, it has been confirmed that not only a purified antigenic protein such as Cryj 1 but also an extract can be used for the oligosaccharide liposome encapsulating the allergen of the present invention.

### Example 9

### Inhibitory Effect by Nasal Administration of Oligosaccharide Liposome Encapsulating Cryj 1 on IgE Production in Mice

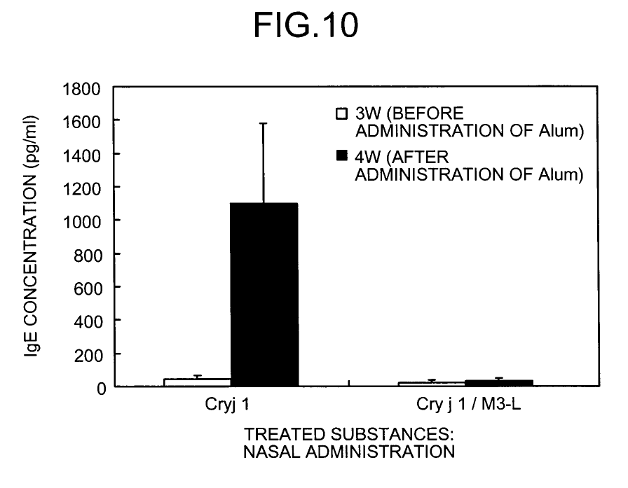

Cryj 1 or the oligosaccharide liposome encapsulating Cryj 1 was administrated nasally three times with one week interval to BALB/c mice aged 6 weeks (the amount of the administered antigen as the protein [total protein amount]: 1 μg/head). One week after the final treatment with the test substance, the mixture of Cryj 1 and alum was intraperitoneally administered to all of the mice (the amount of the administered antigen as the protein: 1 μg/head) to induce the Th2 response. The blood was collected from the orbital cavity before and after the administration with the mixture of Cryj 1 and alum to obtain the serum from each mouse. The amount of total IgE in the obtained serum was measured by EIA (FIG. 10).

The IgE production after the administration with the mixture of Cryj 1 and alum was inhibited in the group treated nasally with the oligosaccharide liposome encapsulating Cryj 1 whereas it could not be inhibited in the group treated nasally with Cryj 1.

In view of the foregoing, it was found that the oligosaccharide liposome encapsulating the pollen antigen by the nasal administration had the effect of inhibiting the IgE production. Therefore, it has been confirmed that the oligosaccharide liposome encapsulating the allergen can give the therapeutic effect by not only the intradermal and subcutaneous administration but also the nasal administration.

### INDUSTRIAL APPLICABILITY

The therapeutic agent for allergy of the present invention has high therapeutic effect as well as the reduced risk for the side effect. Thus, by the use thereof for a hyposensitization therapy, the radical therapy of allergy becomes possible, which is effective at a high rate and safe in a short time period, differently from the conventional hyposensitization therapy.

1. A therapeutic agent for allergy comprising a liposome having on its surface an oligosaccharide which is capable of binding to a lectin derived from an antigen presenting cell and is composed of 2 to 11 sugar residues, and the liposome encapsulating an allergen.

2. The therapeutic agent according to claim 1, wherein the oligosaccharide is composed of 3 to 5 sugar residues.

3. The therapeutic agent according to claim 1 or 2, wherein the oligosaccharide is composed of the sugar residues including mannose.

4. The therapeutic agent according to any one of claims 1 to 3, wherein the allergen is a pollen antigen.

5. The therapeutic agent according to claim 4, wherein the pollen antigen is a cedar pollen antigen.

6. The therapeutic agent according to any one of claims 1 to 5, applying by subcutaneous, intradermal or nasal administration.

7. A liposome having on its surface an oligosaccharide which is capable of binding to a lectin derived from an antigen presenting cell and is composed of 2 to 11 sugar residues, and the liposome encapsulating a pollen antigen.

8. The liposome according to claim 7, wherein the oligosaccharide is composed of 3 to 5 sugar residues.

9. The liposome according to claim 7 or 8, wherein the oligosaccharide is composed of the sugar residues comprising mannose.

10. The liposome according to any one of claims 7 to 9, wherein the pollen antigen is a cedar pollen antigen.
